(19) United States
(12) Patent Application Publication
(10) Pub. No.: US 2015/0283018 A1
(43) Pub. Date: Oct. 8, 2015

(54) Patient Stabilizing Headrest

(71) Applicant: Ricardo A. Aviles, Sierra Vista, AZ (US)

(72) Inventor: Ricardo A. Aviles, Sierra Vista, AZ (US)

(21) Appl. No.: 14/678,722

(22) Filed: Apr. 3, 2015

Related U.S. Application Data
(60) Provisional application No. 61/974,593, filed on Apr. 3, 2014.

(51) Int. Cl. A61G 13/12; A61F 9/007

(52) U.S. Cl. CPC A61G 13/121; A61F 9/007

(57) ABSTRACT

An adjustable device provides support and stabilization for a patient's head during surgical procedures, especially ophthalmologic ones. A frame, including a base and lateral panel, allows an adjustable lateral support and locking mechanism to be engaged with each other and the frame. The adjustable lateral support consists of an elongated member, which is placed through a hole of the lateral panel, and a connected head bracing structure. A first cushioning pad and a second cushioning pad are secured to the base and the head bracing structure, respectively. The locking mechanism uses a spring which pushes a handle against the elongated member. The elongated member traverses through a slot of the handle; when the handle is pressed against the elongated member, the slot applies pressure against the elongated member and prevents movement. The handle can be lifted to allow the elongated member and thus head bracing structure to be repositioned.

The current application claims a priority to the U.S. Provisional Patent application Ser. No. 61/974,593 filed on Apr. 3, 2014.

### FIELD OF THE INVENTION

The present invention relates generally to patient stabilizing headrests. More specifically, the present invention is an adjustable headrest that is compatible with the majority of surgical stretchers.

### BACKGROUND OF THE INVENTION

Eye surgeries require extreme precision and stability due to the complicated and delicate nature of the eye and its surrounding regions. As a result, specialists in the field of ophthalmology are required to complete four years of college, four years of medical school, and four to five years of additional specialized training. One of the problems that surgeons experience during eye surgery is patients turning their heads away from the surgeon; this raises the degree of difficulty for the already highly delicate procedure. Headrests have been invented to combat this problem, but they are limited in their capabilities. One particular solution includes the use of a single block of material that contains a cutout region shaped ideally for the patients head, forcing the head into a single position. This is ideal for the surgeon but is too cumbersome of a solution as it requires a new mold to be created for each patient. Another popular solution is the use of a single cushion that contains a recessed cavity; this provides the perfect vertical support but lacks in lateral constraint and results in the patient having too much horizontal freedom.

The present invention provides a solution for this problem. The present invention contains a gel pad that goes underneath the patients head and utilizes a gel pad in a perpendicular orientation to control the lateral movement of the patient. The distance of the lateral gel pad is adjustable through a lever, resulting in a vertical and lateral head support apparatus compatible with the majority of patients. The present invention not only prevents patients from turning their head during surgery, it also stabilizes the head to further aid the surgeon in the delicate procedure. The headrest is ideal for stabilizing patient's heads for procedures such as cataract surgery, lasik surgery, pterygium removal, eyelid surgery, trabeculoplasty, canaloplasty, cyclocryotherapy, vitrectomy, and other surgical procedures. The preferred embodiment of the present invention is ideal for surgical stretchers which contain two piece split wrist rests, but may be modified to accommodate a multitude of different surgical stretchers. Furthermore, the present invention may be applicable in other medical uses which require variable lateral head support.

### BRIEF DESCRIPTION OF THE DRAWINGS

FIG. 1 is an isometric view of the present invention;

FIG. 2 is a front view thereof;

FIG. 3 is a top view thereof;

FIG. 4 is a right side view thereof;

FIG. 5 is an exploded isometric view thereof;

FIG. 6 is an exploded isometric view thereof depicting the components from a different angle;

FIG. 7 is an isometric view of the present invention on a surgical stretcher; and,

FIG. 8 is a detailed view thereof.

FIG. 9 is a right side view showing operation of a locking mechanism of the present invention.

### DETAIL DESCRIPTIONS OF THE INVENTION

All illustrations of the drawings are for the purpose of describing selected versions of the present invention and are not intended to limit the scope of the present invention.

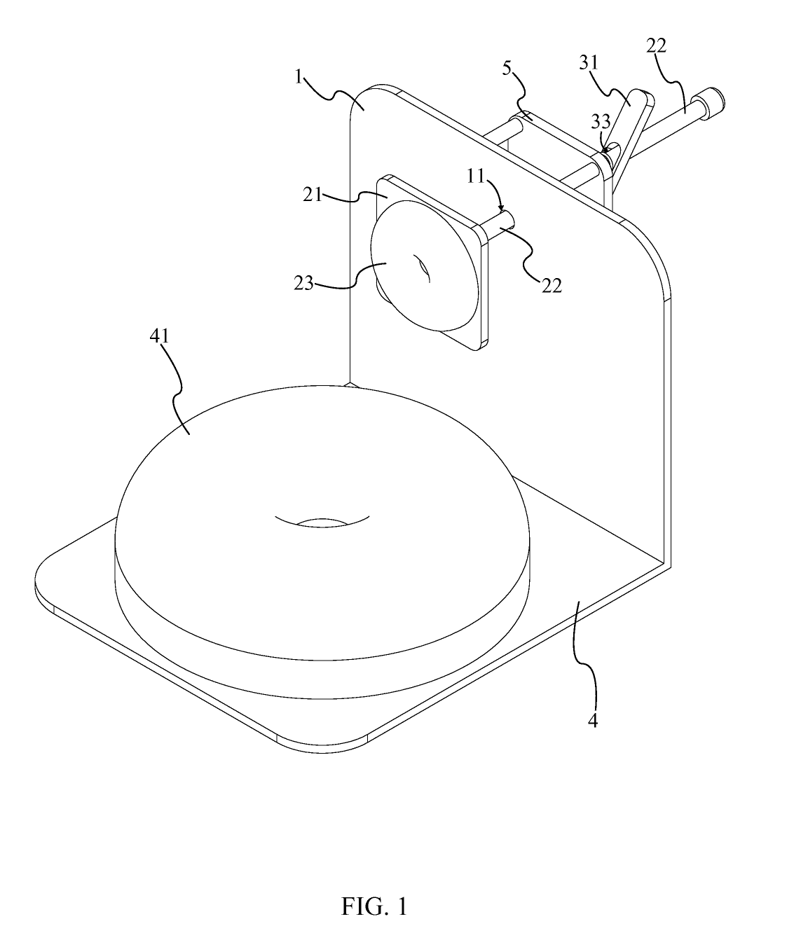

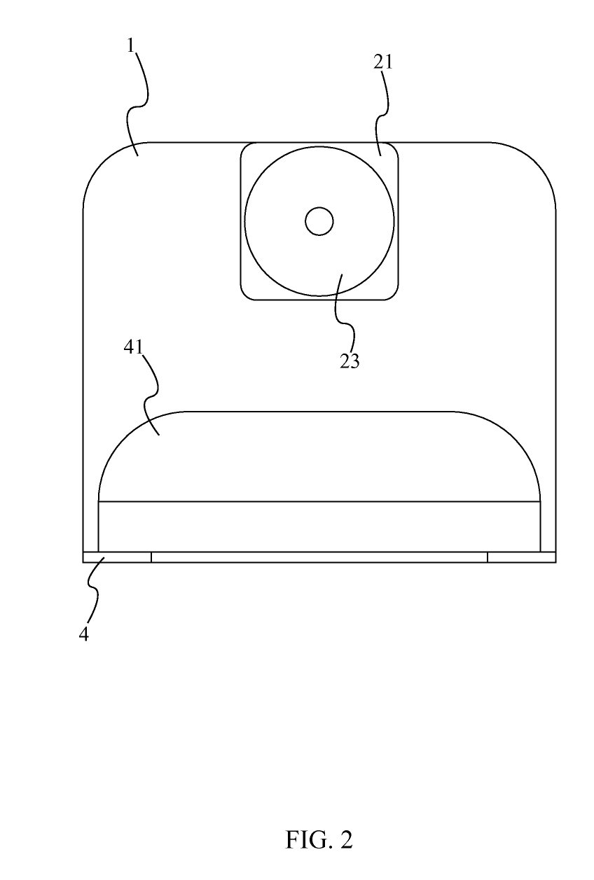

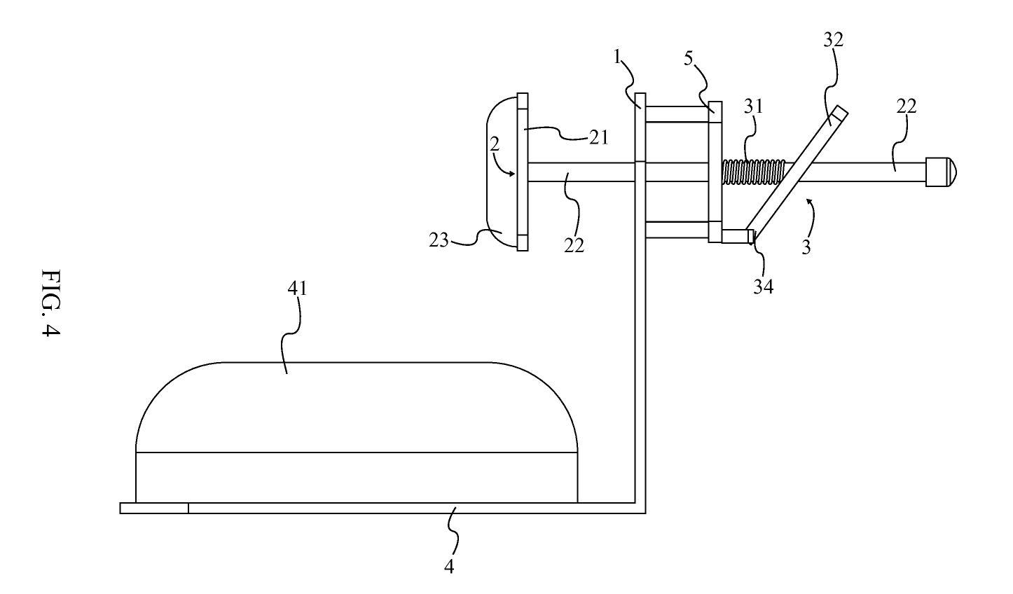

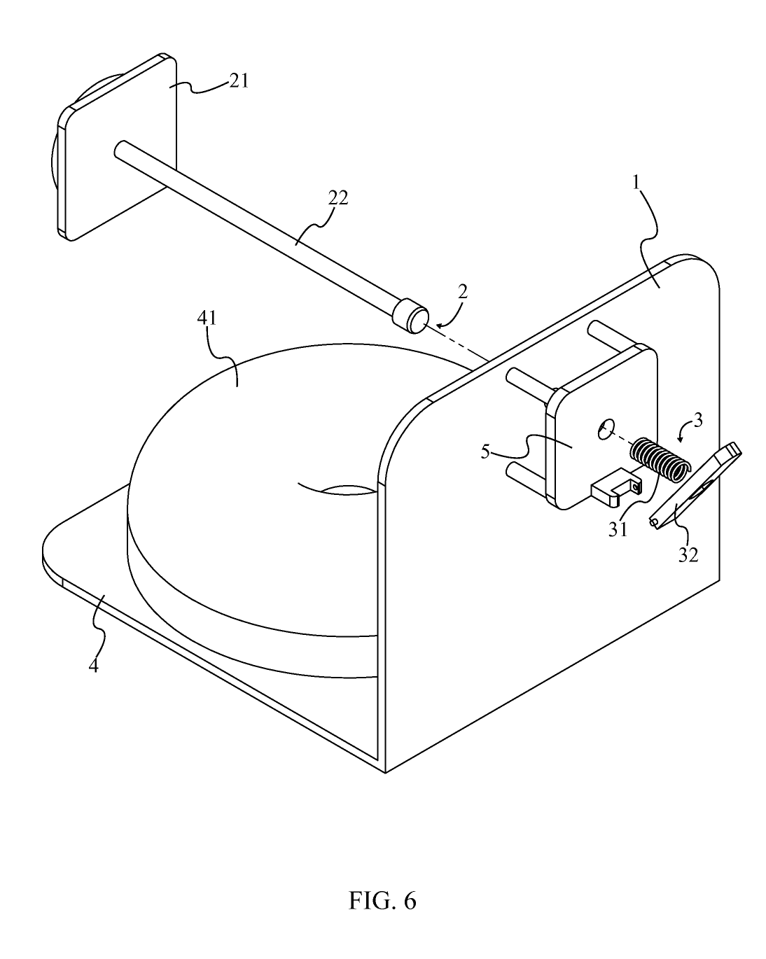

The present invention is a stabilizing headrest with improved means for adjusting the stabilizing headrest to best fit individual patients. The present invention is capable of providing vertical and horizontal support for a patient's head. The present invention comprises a frame which supports an adjustable lateral support 2 and a locking mechanism 3 which enables locking of the adjustable lateral support 2 in a desired position. The frame itself comprises a lateral panel 1, with which the adjustable lateral support 2 is engaged. The adjustable lateral support 2 comprises a head bracing structure 21 and an elongated member 22; the head bracing structure 21 provides the horizontal support for a patient's head while the elongated member 22 allows the head bracing structure 21 to be moved to a desired position. The locking mechanism 3 comprises a spring 31 and a handle 32, which the elongated member 22 traverses through; the spring 31 and handle 32 interact with the elongated member 22 in order to lock the elongated member 22 in a desired position and prevent unintentional movement. Components and configurations of the present invention are illustrated via FIG. 1-FIG. 9.

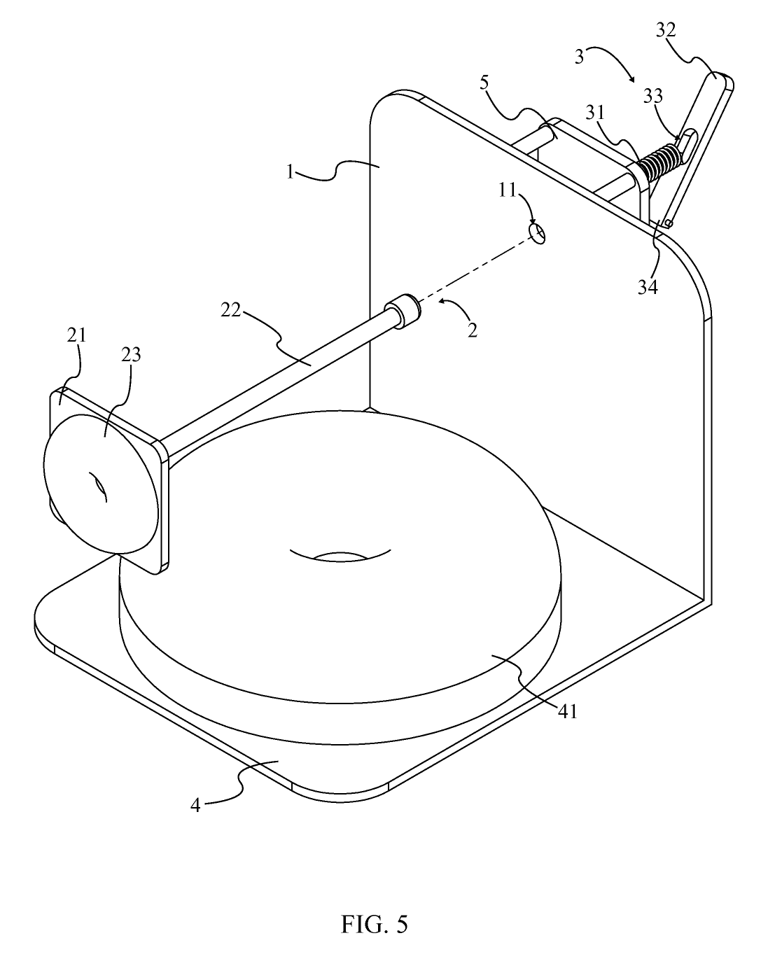

The lateral panel 1 comprises a hole 11 which receives the elongated member 22. As the elongated member 22 is slidably engaged with the hole 11, the elongated member 22 can be moved along a linear path. Movement of the elongated member 22 results in corresponding movement of the head bracing structure 21, allowing a user to adjust the position of the head bracing structure 21 relative to a patient's head. In short, the adjustable lateral support 2 is capable of reciprocal motion through the hole 11 of the lateral panel 1. The lateral panel 1 and hole 11 are best seen in FIG. 1 and FIG. 5.

A cap can be provided at a free end of the elongated member 22, opposite the head bracing structure 21. This cap, larger than the hole 11 of the lateral panel 1, prevents the elongated member 22 from completely sliding out of the hole 11, which would effectively disengage the adjustable lateral support 2 from the frame of the present invention. The cap can be any feature that is larger than the hole 11 and thus prevents the free end of the elongated member 22 from sliding through the hole 11.

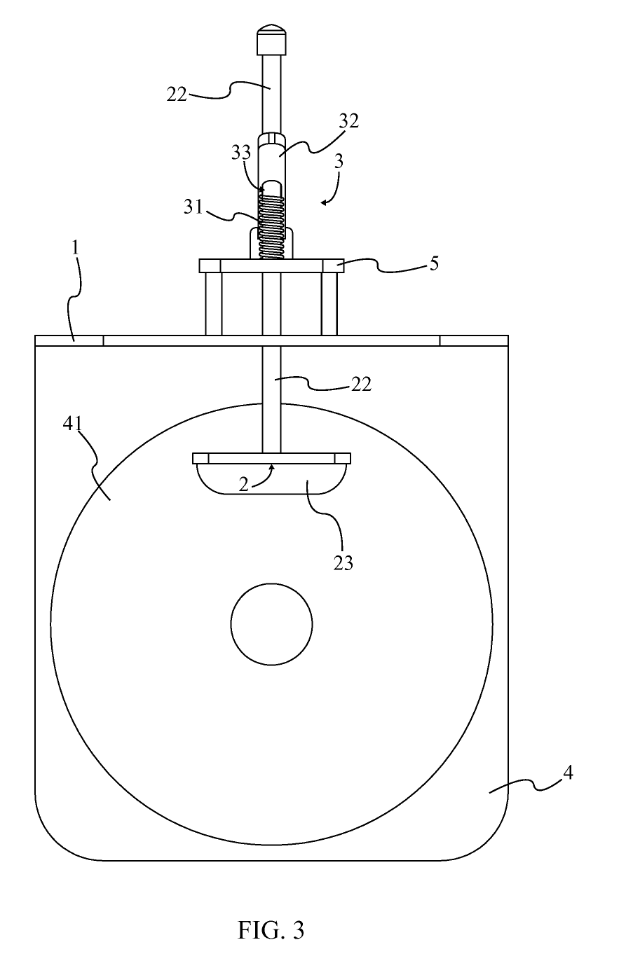

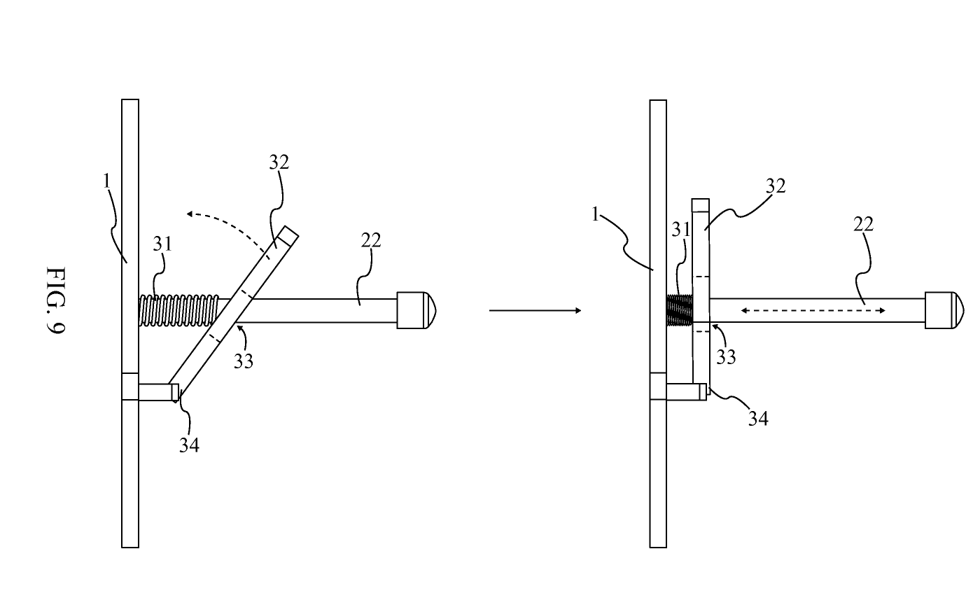

The locking mechanism 3, which is positioned on the other side of the lateral panel 1 as compared to the head bracing structure 21, is capable of interacting with the elongated member 22. Supporting this, the spring 31 is helically positioned around the elongated member 22, between the lateral panel 1 and the handle 32. The handle 32 itself comprises a slot 33, through which the elongated member 22 passes. The handle 32 is capable of rotating about a fixed end 34, as the handle 32 is hingedly connected to the lateral panel 1. As a result, the spring 31 pushes against the handle 32, rotating the handle 32 towards the elongated member 22. The resulting pressure effectively locks the elongated member 22 in place, as a rim of the slot 33 is pressed against the elongated member 22. An outside force, such as applied by a user, may push the handle 32 against the spring 31 in order to rotate it about the fixed end 34 and lift it from the elongated member 22. This unlocks the elongated member 22, allowing its position (and correspondingly the position of the head bracing support) to be adjusted. The locking mechanism 3 is best illustrated in FIG. 3-FIG. 6, as well as FIG. 9 which provides a visual example of how the locking mechanism 3 is operated to allow or prevent the elongated member 22 from being moved along a linear path.

Effectively, the handle 32 acts as a lever, with a fixed rotation point connected to the lateral panel 1 and torque being applied by the spring 31. An opposing force (e.g.

applied by a user) can overcome the torque generated by the spring 31 and rotate the handle 32 away from the elongated member 22. The combination of the handle 32 with the elongated member 22 allows the head bracing support to easily be adjusted to accommodate individual characteristics of different patients.

Potentially, in one embodiment the elongated member 22 comprises a plurality of handle 32-receiving ridges which are positioned along the elongated member 22. These handle 32-receiving ridges improve the contact between the handle 32 and the elongated member 22, resulting in a more secure locking of the elongated member 22 as compared to an embodiment without handle 32-receiving ridges.

Preferably, the present invention comprises a base 4, which expands the frame as earlier introduced. The base 4 serves two purposes, supporting the other components of the present invention as well as serving as an addition bracing body for the back of a patient's head (similar to how the head bracing structure 21 provides horizontal support for a patient's head). In order to provide sufficient room for a person's head, the lateral panel 1 is adjacently connected to the base 4. The lateral panel 1 shares an edge with the base 4, analogous to a wall and floor. The head bracing structure 21 is positioned on the same side of the lateral panel 1 as the base 4, as necessary to provide support for a patient's head. In order to offer a more ergonomic support, a first cushion and second cushion can be utilized in combination with the base 4 and the adjustable lateral support 2.

The first cushioning pad 41, provided to support the back of patient's head, is connected adjacent to the base 4. The second cushioning pad 23, which is provided to support the side of a patient's head, is connected adjacent to the head bracing structure 21. These cushioning pads, illustrated in FIG. 1-FIG. 8, serve as a more comfortable surface which directly contacts the patient's head. The pads are illustrated in a torus configuration, but can be implemented in a variety of shapes. Likewise, the cushioning aspect of the pads can be implemented in a number of ways. One example includes inflating a volume within each cushioning pad, which allows the hardness of each pad to be adjusted by increasing or decreasing the air pressure within the pad; as a result, the support provided by the pads can be better adjusted to meet the preferences of different patients. Other examples of ways the cushioning pads can be implemented include the use of a gel-like composition such as a polyurethane gel, feathers, foam, water, and gel beads. It is noted that the first cushioning pad 41 and second cushioning pad 23 do not need to be made from the same materials or have the same shape.

As the head bracing structure 21 serves as a connection point for both the elongated member 22 and the head bracing structure 21, it preferably has a shape which is fitted to both. The illustrations show the head bracing structure 21 being shaped as a planar body, oriented to be parallel with the lateral panel 1. While the head bracing structure 21 can be angled with respect to the lateral panel 1, a parallel configuration is preferable in order to better brace the side of a patient's head.

The manner in which the first cushioning pad 41 is connected to the base 4 and the second cushioning pad 23 is connected to the head bracing structure 21 is not restricted by the present invention. For example, depending on manufacturer preference, glue, self adhesive tape, double sided tape, and fasteners such as bolts and screws can be used to secure the cushioning pads to their respective structures. In one embodiment, the first cushioning pad 41 is not connected to the base 4 but is instead simply placed atop the base 4. Such an embodiment allows a surgeon to freely adjust the position of the first cushioning pad 41 with respect to the base 4, allowing adjustments to be made for increased patient comfort.

Potentially, an offset plate 5 can be adjacently connected to the back of the lateral panel 1. Positioned on the opposite side of the lateral panel 1 as the head bracing structure 21, the offset plate 5 serves as an alternative connection point for the spring 31 and handle 32. While this results in some minor changes, for example allowing the use of a smaller spring 31, the offset plate is not necessary; the basic configuration and operation of the present invention is not negatively impacted by omission of the offset plate 5.

Provided the offset plate 5 is utilized, the spring 31 is positioned between the offset plate 5 and the handle 32, as opposed to another embodiment where the spring 31 is positioned between the lateral panel 1 and the handle 32.

To help support the spring 31, it can be positioned around the elongated member 22. This helps to minimize the effects of other forces (for example, gravity), allowing the spring 31 to compress and extend along a single linear path. This prevents the handle 32 from pressing down on the spring 31, which would decrease user friendliness of the present invention. Ultimately, an embodiment where the spring 31 is not positioned around the elongated member 22 is possible, though such a configuration is not preferable for the reasons previously stated.

Recapping the present invention, the elongated member 22 traverses through the hole 11 of the lateral panel 1, with the head bracing structure 21 being connected to one end of the elongated member 22, such that the head bracing structure 21 is positioned over the base 4. The locking mechanism 3 is positioned on the opposite side of the lateral panel 1 as the head bracing structure 21. The spring 31 and handle 32 of the locking mechanism 3 are connected to the lateral panel 1, the handle 32 being connected at a fixed end 34 in a manner that allows it to rotate. The elongated member 22 traverses through a slot 33 of the handle 32, resulting in the spring 31 pressing the handle 32 against the elongated member 22. A rim of the slot 33 contacts the elongated member 22, creating an applied force that prevents movement of the elongated member 22.

To disengage the handle 32 from the slot 33, a user simple pushes the handle 32 against the spring 31, causing the spring 31 to compress and the slot 33 of the handle 32 to be lifted from the elongated member 22. As the handle 32 is no longer applying force against the elongated member 22, the elongated member 22 can freely be moved along an axis shared with the hole 11 of the lateral panel 1. A user can then adjust the position of the head bracing structure 21 to better support a patient's head.

Once the head bracing structure 21 has been repositioned as desired, a user can release the handle 32. The spring 31 then extends in order to return to its equilibrium position. As the spring 31 extends, it presses against the handle 32 and causes the handle 32 to rotate into the elongated member 22. The rim of the slot 33 applies a force against the elongated member 22, locking the elongated member 22 in place and prevents undesired movement. When combined with a base 4, corresponding first cushioning pad 41, and a second cushioning pad 23 for the head bracing structure 21, the present invention provides a patient stabilizing headrest, for example as used in ophthalmological surgeries.

Other actuating and locking mechanisms 3 may be utilized in alternative embodiments of the present invention. For example, a slot key configuration might be utilized to adjust the lateral distance of the lateral panel 1 and therefore the second cushioning pad 23. In yet another embodiment, threaded rods connect the lateral panel 1 with the base 4, which can be rotated in order to adjust the distance between the lateral panel 1 and base 4.

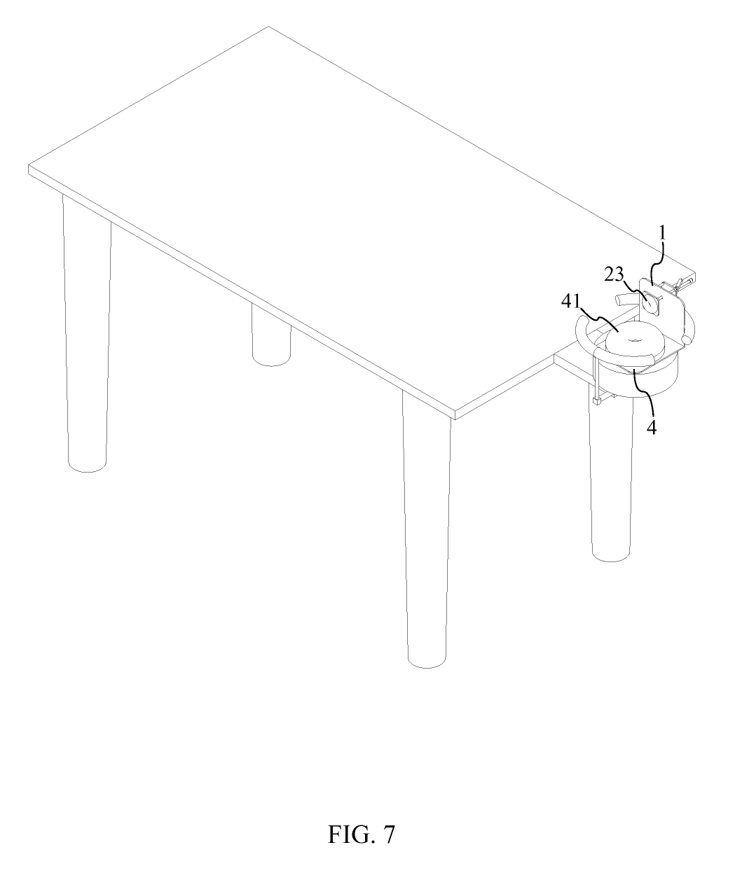

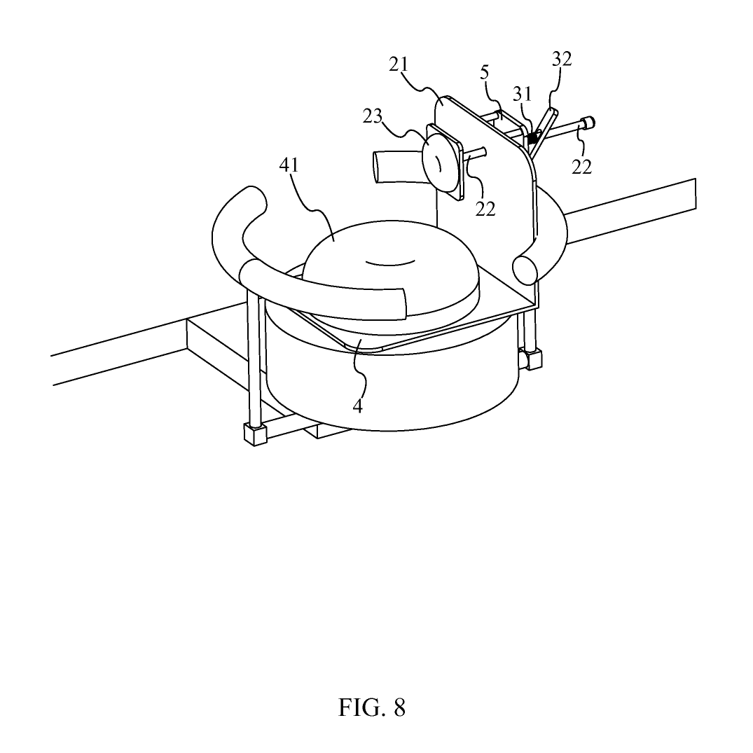

In the preferred embodiment, the present invention is utilized in conjunction with split-style wrist rests as seen in FIG. 7-8. The color and pattern scheme, material composition, and the dimensions are all subject to change in the final embodiment of the present invention.

Although the invention has been explained in relation to its preferred embodiment, it is to be understood that many other possible modifications and variations can be made without departing from the spirit and scope of the invention as hereinafter claimed.

What is claimed is:

1. A patient stabilizing headrest comprises:
a lateral panel;
an adjustable lateral support;
a locking mechanism;
the adjustable lateral support comprises a head bracing structure and an elongated member;
the locking mechanism comprises a spring and a handle;
the lateral panel comprises a hole;
the handle comprises a slot;
the elongated member being adjacently connected to the head bracing structure;
the hole traversing through the lateral panel;
the spring being positioned adjacent to the handle;
the elongated member being slidably engaged with the hole; and
the elongated member traversing through the slot.

2. The patient stabilizing headrest as claimed in claim 1 comprises:
a fixed end of the handle being hingedly connected to the lateral panel.

3. The patient stabilizing headrest as claimed in claim 1 comprises:
a base; and
the lateral panel being perpendicularly and adjacently connected to the base.

4. The patient stabilizing headrest as claimed in claim 2 comprises:
a first cushioning pad adjacently connected to the base; and
the first cushioning pad being centrally positioned on the base.

5. The patient stabilizing headrest as claimed in claim 1 comprises:
a second cushioning pad adjacently connected to the head bracing structure;
the head bracing structure being positioned between the second cushioning pad and the elongated member; and
the second cushioning pad being centrally positioned on the head bracing structure.

6. The patient stabilizing headrest as claimed in claim 1 comprises:
the head bracing structure being a rigid planar body; and
the rigid planar body being parallel with the lateral panel.

7. The patient stabilizing headrest as claimed in claim 1 comprises:
an offset plate;
the offset plate being adjacently connected to the lateral panel; and
the lateral panel being positioned between the head bracing structure and the offset plate.

8. The patient stabilizing headrest as claimed in claim 7 comprises:
a fixed end of the handle being hingedly connected to the offset plate;
the spring being connected between the offset plate and the handle; and
the spring being helically positioned around the elongated member.

9. The patient stabilizing headrest as claimed in claim 1 comprises:
the spring being connected between the lateral panel and the handle; and
the spring being helically positioned around the elongated member.

10. A patient stabilizing headrest comprises:
a lateral panel;
an adjustable lateral support;
a locking mechanism;
a base;
the adjustable lateral support comprises a head bracing structure and an elongated member;
the locking mechanism comprises a spring and a handle;
the lateral panel comprises a hole;
the handle comprises a slot;
the elongated member being adjacently connected to the head bracing structure;
the hole traversing through the lateral panel;
the spring being positioned adjacent to the handle;
the elongated member being slidably engaged with the hole;
the elongated member traversing through the slot;
a fixed end of the handle being hingedly connected to the lateral panel; and
the lateral panel being perpendicularly and adjacently connected to the base.

11. The patient stabilizing headrest as claimed in claim 10 comprises:
a first cushioning pad adjacently connected to the base; and
the first cushioning pad being centrally positioned on the base.

12. The patient stabilizing headrest as claimed in claim 10 comprises:
a second cushioning pad adjacently connected to the head bracing structure;
the head bracing structure being positioned between the second cushioning pad and the elongated member; and
the second cushioning pad being centrally positioned on the head bracing structure.

13. The patient stabilizing headrest as claimed in claim 10 comprises:
the head bracing structure being a rigid planar body; and
the rigid planar body being parallel with the lateral panel.

14. The patient stabilizing headrest as claimed in claim 10 comprises:
an offset plate;
a fixed end of the handle being hingedly connected to the offset plate;
the offset plate being adjacently connected to the lateral panel;
the lateral panel being positioned between the head bracing structure and the offset plate;
the spring being connected between the offset plate and the handle; and
the spring being helically positioned around the elongated member.

15. The patient stabilizing headrest as claimed in claim 10 comprises:
the spring being connected between the lateral panel and the handle; and
the spring being helically positioned around the elongated member.
